(19) United States
(12) Patent Application Publication
(10) Pub. No.: US 2026/0083962 A1
(43) Pub. Date: Mar. 26, 2026

(54) Spinal Cord Stimulator Lead Migration Arrest And Correction System

(71) Applicant: Corrective Stim LLC, Savannah, GA (US)

(72) Inventor: Andrew Pandya, Savannah, GA (US)

(73) Assignee: Corrective Stim LLC, Savannah, GA (US)

(21) Appl. No.: 18/891,827

(22) Filed: Sep. 20, 2024

(51) Int. Cl. A61N 1/05

(52) U.S. Cl. CPC A61N 1/0558

(57) ABSTRACT

A spinal cord stimulator lead migration arrest and correction system to mitigate lead migration during spinal cord stimulation trials or subsequent to spinal cord stimulator implantation.

### I. FIELD OF THE INVENTION

A spinal cord stimulator lead migration arrest and correction system to mitigate lead migration during spinal cord stimulation trials or subsequent to spinal cord stimulator implantation.

### II. BACKGROUND OF THE INVENTION

A spinal cord stimulator is an implanted device that sends low levels of pulsed electricity directly into the spinal cord to relieve pain. Spinal cord stimulators include thin wires, referred to as “electrodes”, and a small battery pack, referred to as the “generator”. The electrodes are placed between the spinal cord and the vertebrae in the epidural space, and the generator is implanted under the skin.

Before the generator is implanted under the skin, a trial period is conducted in which the electrodes are temporarily implanted in the epidural space of the spine and the generator is located outside the body. The trial period typically lasts for a period of about one week. If the electrodes move from their original location during the trail, referred to as “lead migration”, the spinal cord stimulator may not block pain or may not block pain as effectively.

Lead migration is the most commonly reported complication of spinal cord stimulation procedures and lead migration during trials of spinal cord stimulation can compromise both the success of the trial as well as the efficacy of subsequent implantation. Studies show that on average there is about 28 millimeter or 1.17 vertebral body levels of lead migration during spinal cord stimulation trials. If spinal cord lead migration occurs during a trial period, then a follow-up surgery will be required to place the electrodes back in the proper location in the epidural space and commencement of a re-trial period. In instances where the spinal cord stimulator has been implanted, spinal cord lead migration requires a complete follow up revision surgery. Thus, lead migration during spinal cord stimulation trials and after implantation of the spinal cord stimulator presents a significant challenge for clinical care.

Accordingly, there is a long felt but unresolved need for devices and techniques to arrest and/or correct lead migration during spinal cord stimulation trials and subsequent to spinal cord stimulator implantation. There would be a substantial advantage during spinal cord stimulation trials if there was a spinal cord stimulator lead migration arrest and correction apparatus that can arrest and allow ready correction of lead migration during spinal cord stimulation trials. Additionally, there would be further substantial advantages in the use of particular elements of the apparatus in the final implantation of the spinal cord stimulator.

### III. SUMMARY OF THE INVENTION

Accordingly, a broad object of particular embodiments of the invention can be to provide an apparatus that mitigates and/or allows correction of lead migration during spinal cord stimulation trials including one or more of: an electrode sheath implantable in an interspinal tissue of a subject, wherein the electrode sheath has a sheath interior surface defining an interior space between a sheath inlet end and a sheath outlet end configured to receive a spinal cord stimulator electrode with the electrode lead extending from the sheath outlet end for placement of the electrode lead in an epidural space of the subject; a pouch coupled to the sheath inlet end and extending to a pouch open end, wherein the pouch has a configuration to enclose within an electrically conductive interface coupled to the spinal cord stimulator electrodes; and a receptacle having mateable portions engageable to define a receptacle exterior surface adapted to affix the receptacle to the subject proximate an implant location of the electrode sheath and a receptacle interior space extending between a receptacle inlet port and a receptacle outlet port open to the receptable exterior surface, wherein the pouch enclosing the electrically conductive interface can be disposed within the receptacle with the pouch coupled to the sheath inlet end of the electrode sheath extending through the receptacle outlet port to allow implantation of the electrode sheath. A pulse generator can be disposed outside of the receptacle, wherein the pulse generator can be removably electrically coupled to the electrically conductive interface within the pouch by a pulse generator cord extending through the receptacle inlet port. In particular embodiments a cushion wrap can be removably disposed about the receptacle to absorb forces received by the receptacle.

Another broad object of particular embodiments of the invention can be to provide a method of making an apparatus that mitigates and allow correction of lead migration during spinal cord stimulation trials including one or more of: configuring an electrode sheath to implant in an interspinal tissue of a subject, wherein the electrode sheath has a sheath interior surface defining an interior space between a sheath inlet end and a sheath outlet end configured to receive a spinal cord stimulator electrode with the electrode lead extending from the sheath outlet end for placement of the electrode lead in an epidural space of the subject; and coupling a pouch to the sheath inlet end and which extends to a pouch open end, wherein the pouch has a configuration to enclose within an electrically conductive interface coupled to the one or more spinal cord stimulator electrodes; and configuring mateable portions of a receptacle which engage to define a receptacle exterior surface adapted to affix the receptacle to the subject proximate an implant location of the electrode sheath and a receptacle interior space extending between a receptacle inlet port and a receptacle outlet port open to said receptable exterior surface, wherein pouch enclosing said electrically conductive interface disposed within said receptacle, said pouch coupled to said sheath inlet end of said electrode sheath extending through said receptacle outlet port to allow implantation of said electrode sheath. The method can further include one or more of: disposing a pulse generator disposed outside of said receptacle, wherein the pulse generator can be removably electrically coupled to the electrically conductive interface by a pulse generator cord extending through said receptacle inlet port; and removably disposing a cushion wrap about said receptacle to absorb forces received by said receptacle.

Another broad object of particular embodiments of the invention can be to provide a method of using an apparatus that mitigates and/or allows ready correction of lead migration during spinal cord stimulation trials including one or more of: disposing one or more spinal cord stimulator electrodes through an interior space of an electrode sheath between a sheath inlet end and a sheath outlet end with said one or more spinal cord electrodes extending from said sheath outlet end for placement of one or more electrode leads in an epidural space of a subject; implanting said electrode sheath in an interspinal tissue of said subject; enclosing an electrically conductive interface coupled to said one or more spinal cord stimulator electrodes inside of a pouch coupled to the electrode sheath inlet end; disposing the pouch enclosing the electrically conductive interface within a receptacle, wherein the pouch coupled to the sheath inlet end of the electrode sheath extending through a receptacle outlet port; removably electrically coupling a pulse generator disposed outside of the pouch to the electrically conductive interface by a pulse generator cord extending through said receptacle inlet port; and removably disposing a cushion wrap about said receptacle to absorb forces received by said receptacle.

Naturally, further objects of the invention are disclosed throughout other areas of the specification, drawings, photographs, and claims.

### IV. BRIEF DESCRIPTION OF THE DRAWINGS

FIG. 1 is a perspective view illustrating a method of using a particular embodiment of the spinal cord stimulator lead migration arrest and correction apparatus with a spinal cord stimulator having the spinal cord stimulator electrode implanted in the epidural space of a subject.

FIG. 2 is side view illustrating a method of using a particular embodiment of the spinal cord stimulator lead migration arrest and correction apparatus with a spinal cord stimulator having the spinal cord stimulator electrode implanted in the epidural space of a subject.

FIG. 3 is side elevation view of an embodiment of an electrode sheath.

FIG. 4 is a cross section 4-4 of the electrode sheath shown in FIG. 3.

FIG. 5 is an illustration of a particular embodiment of the spinal cord stimulator lead migration arrest and correction apparatus including a pouch enclosing an electrically conductive interface coupled to the one or more spinal cord stimulator electrodes which extend through an electrode sheath coupled to the pouch.

FIG. 6 is a cross-section view of a particular embodiment of the spinal cord stimulator lead migration arrest and correction apparatus including a receptacle configured to receive the pouch enclosing an electrically conductive interface with the electrode sheath and a portion of the pouch extending through a receptacle outlet port and the pulse generator cord extending through the receptacle inlet port with the pulse generator external to the receptacle.

FIG. 7 is a cross-section view of a particular embodiment of the spinal cord stimulator lead migration arrest and correction apparatus including a receptacle configured to receive the pouch enclosing an electrically conductive interface and the pulse generator with the electrode sheath and a portion of the pouch extending through a receptacle outlet port.

FIG. 8 is a top view of a particular embodiment of the spinal cord stimulator lead migration arrest and correction apparatus including the receptacle containing the pouch enclosing the electrically conductive interface with the electrode sheath and a portion of the pouch extending through a receptacle outlet port.

FIG. 9 is a side elevation view of a particular embodiment of the spinal cord stimulator lead migration arrest and correction apparatus including the receptacle containing the pouch enclosing the electrically conductive interface with the electrode sheath and a portion of the pouch extending through a receptacle outlet port.

FIG. 10A is a side elevation view of a particular embodiment of the spinal cord stimulator lead migration arrest and correction apparatus including the receptacle containing the pouch enclosing the electrically conductive interface with the electrode sheath and a portion of the pouch extending through a receptacle outlet port including a particular embodiment of a cushion wrap surrounding the receptacle.

FIG. 10B is an illustration of a particular embodiment of the cushion wrap having mateable peripheral margin ends adjustable to secure the cushion wrap about the receptacle.

FIG. 10C is an illustration of an alternate embodiment of the mateable peripheral margin ends adjustable to secure the cushion wrap about the receptacle.

FIG. 10D is an illustration of a particular embodiment of the cushion wrap having an elastic element coupled to the cushion wrap peripheral margin to secure the cushion wrap about the receptacle.

FIG. 10E is an enlarged cross section 10E-10E depicting the elastic element disposed in the peripheral margin of the cushion wrap.

FIG. 11 is an illustration of a method of implanting an electrode sheath in a subject concurrent with implantation of a spinal cord stimulator in a subject.

### V. DETAILED DESCRIPTION OF THE INVENTION

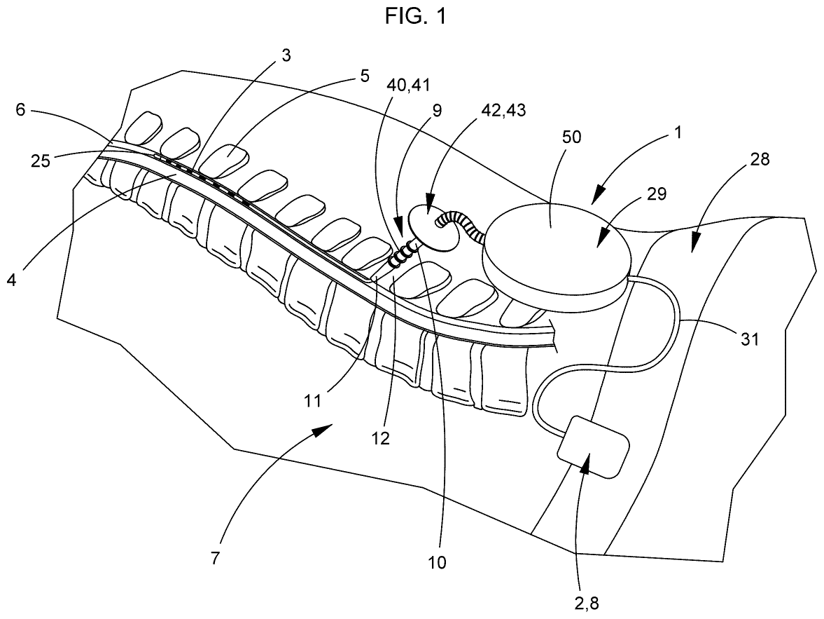

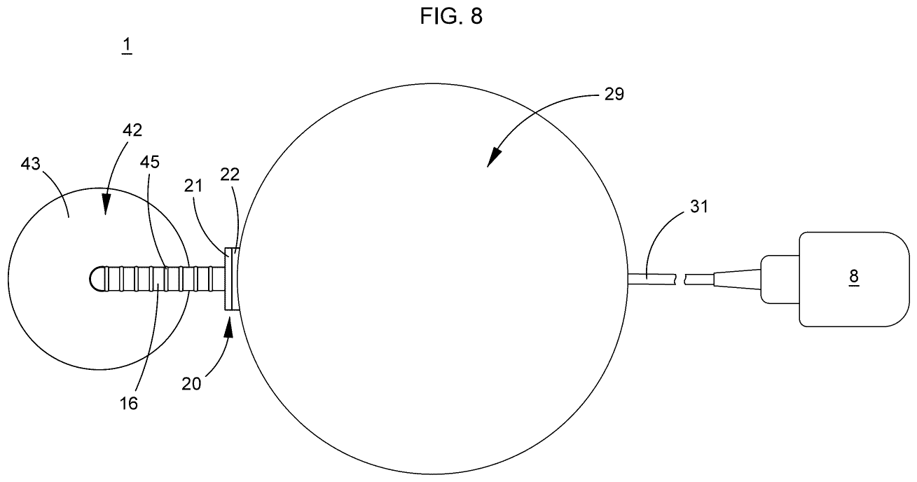

Generally, referring to FIGS. 1 through 10A-10E, the invention provides embodiments of a spinal cord stimulator lead migration mitigation apparatus (1) useful to arrest and correct lead migration during spinal cord stimulation trials, methods of making the spinal cord stimulator lead migration mitigation apparatus (1), and methods of using the spinal cord stimulator lead migration mitigation apparatus (1).

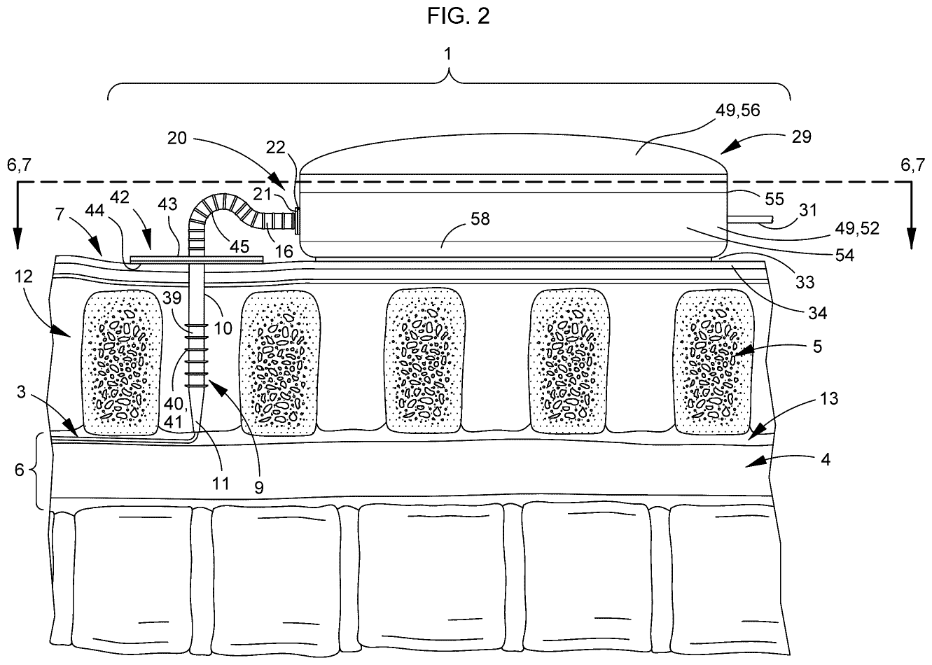

Now, with primary reference to FIGS. 1 through 2, depicting a method of using particular embodiments of the inventive apparatus (1) in the context of a spinal cord stimulator trial period in which a spinal cord stimulator (2) has the spinal cord stimulator electrode (3) temporarily placed between the spinal cord (4) and the vertebrae (5) in the epidural space (6) of a subject (7) with the pulse generator (8) remaining outside the subject (7) during the spinal cord stimulator trial. To maintain sterility of components of the spinal cord stimulator (2) that remain outside of the subject (7), the spinal cord stimulator electrode (3) can be pass within an electrode sheath (9) between an electrode sheath inlet end (10) and an electrode sheath outlet end (11) with the spinal cord electrode (3) extending from the electrode sheath outlet end (11) for placement in the epidural space (6) of the subject (7). The electrode sheath (9) can be implanted in the interspinal tissue (12) of the subject (7) to facilitate placement of the spinal cord stimulator electrode (3) in the epidural space (6) of the subject (7). In particular instances, the electrode sheath outlet end (11) can be disposed proximate ligament flavum (13).

Now, with primary reference to FIGS. 1 through 5, the spinal cord stimulator electrode (3) can be connected to an electrically conductive interface (14). The electrically conductive interface (14) coupled to the spinal cord stimulator electrode (3) can be disposed within a pouch (15) coupled directly, or indirectly with flexible conduit (16), to the electrode sheath inlet end (10). The pouch (15) can provide a pouch internal surface (17), which upon closure of a pouch open end (18) as illustrated, can enclose a sterile interior space (19). In particular embodiments, the pouch (15) can be removably coupled to the electrode sheath inlet end (10) or the flexible conduit (16) by operation of an electrode sheath connector (20) having mateable connector parts (21, 22). In particular embodiments, the mateable connector parts (21, 22) can include a first connector part (21) coupled directly or indirectly to the sheath inlet end (10) and a second connector part (22) coupled to the pouch (15), wherein, the first connector part (21) removably mates to the second connector part (22) to couple the electrode sheath (9) to the pouch (15). The electrically conductive interface (14) disposed within the pouch (15) can include an electrically conductive interface connector (23) configured to electrically couple to the pulse generator (8) which can be disposed outside of the pouch (15). The pouch (15) can include a connector access port (24) adapted or configured to seal around the conductive interface connector (23). The pulse generator (8) can be removably electrically coupled to the electrically conductive interface (14) via the electrically conductive interface connector (23). In particular embodiments, the pouch (15) can be adhered to the subject (7) in fixed spatial relation to the implanted electrode sheath (9) to minimize movement of the implanted electrode sheath (9) and the electrode lead (25) in the epidural space (6) of the subject (7). In certain variants of the invention, a pouch biocompatible adhesive layer (26) can be disposed on the pouch (15). A pouch biocompatible adhesive peelable layer (27) can cover the biocompatible adhesive layer (26). The pouch peelable layer (27) can be manually peeled away to expose the pouch biocompatible adhesive layer (26) to adhere the pouch (15) to the subject (7). The pulse generator (8) can be located proximate the subject (7), affixed directly to the subject (7), or affixed to an article (28) worn by the subject (7).

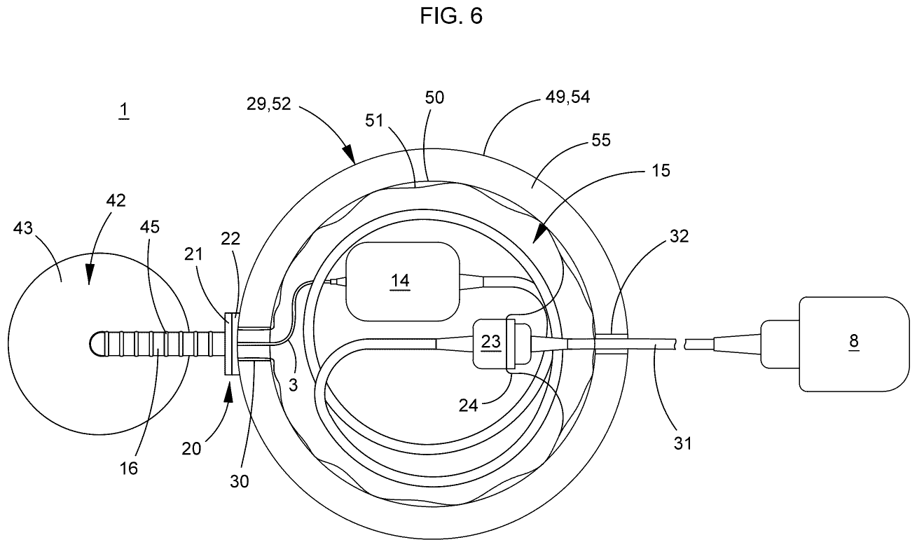

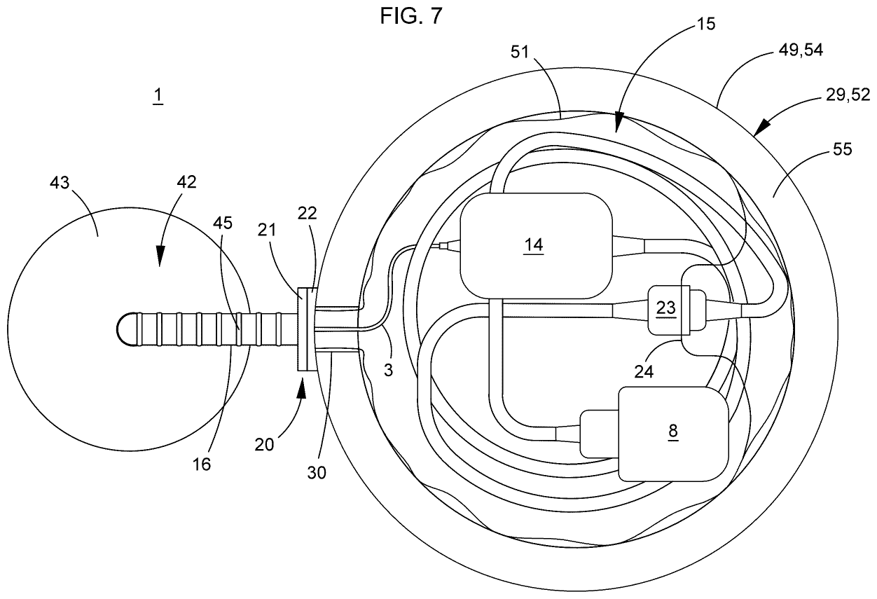

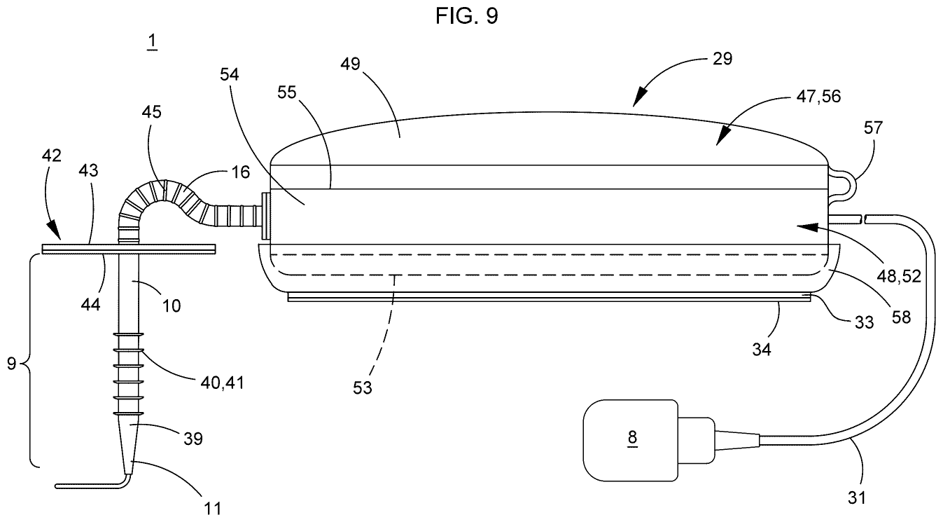

Now, with primary reference to FIGS. 6 through 9, in particular embodiments, the pouch (15) enclosing the electrically conductive interface (14) can be placed within a receptacle (29) with a portion of the pouch (15) or the flexible conduit (16) coupled to the electrode sheath inlet end (10) of the electrode sheath (9) extending through a receptacle outlet port (30). The pulse generator (8) can be removably electrically coupled to the electrically conductive interface (14) by a pulse generator cord (31) extending through a receptacle inlet port (32) (as shown in the example of FIG. 6). In certain instances, the pulse generator (8) can be disposed in the receptacle (29) (as shown in the example of FIG. 7). In particular embodiments, the receptacle (29) can be adhered to the subject (7) in fixed spatial relation to the electrode sheath (9) implanted in subject (7) to mitigate movement of the implanted electrode sheath (9) and migration of the electrode lead (25) in the epidural space (6) of the subject (7) (as shown in the example of FIGS. 1 and 2). In particular embodiments, a resiliently flexible layer (58) can be coupled to the receptacle (29) (as shown in the example of FIG. 9). In certain variants of the invention, a receptacle biocompatible adhesive layer (33) can be disposed on the receptacle (29) (as shown in the example of FIG. 9). A receptacle biocompatible adhesive peelable layer (34) can cover the receptacle biocompatible adhesive layer (33). The receptacle biocompatible adhesive peelable layer (34) can be manually peeled away to expose the receptacle biocompatible adhesive layer (33).

Again, with primary reference to FIGS. 1 through 4, in particular embodiments, the pouch (15) can be uncoupled from the electrode sheath inlet end (10) by uncoupling the first connector part (21) coupled directly or indirectly to the sheath inlet end (20) from the second connector part (22) coupled to the pouch (15). The first connector part (21) uncoupled from the second connector part (22) allows the electrode sheath (9) to remain implanted in the subject (7) with the pouch (7) moved a sufficient distance from the implanted electrode sheath (9) to allow manipulation of the spinal cord stimulator electrode (3) to adjust placement of the spinal cord stimulator lead (25) in the epidural space (6) of the subject (7). The placement of the spinal cord stimulator electrode lead (25) can be adjusted to improve placement of the spinal cord stimulation electrode lead (25), correct placement of the spinal cord stimulation electrode lead (25) due to migration, or to exchange the spinal cord stimulator electrical interface (14).

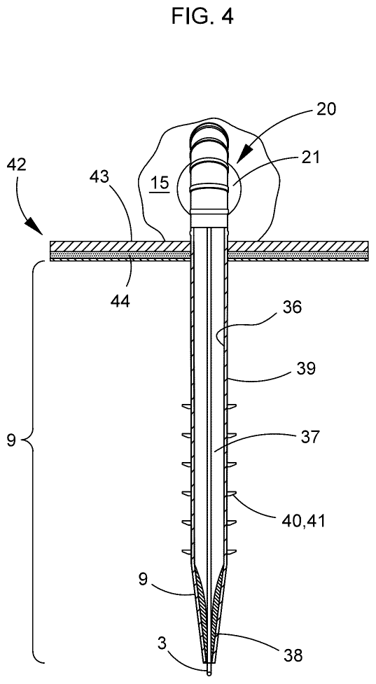

Again, with primary reference to FIGS. 1 through 4, particular embodiments of the apparatus (1) including an electrode sheath (9) implantable in an interspinal tissue (12) of a subject (7). The electrode sheath (9) can have an electrode sheath internal surface (36) defining a sheath interior space (37) between an electrode sheath inlet end (10) and an electrode sheath outlet end (11) adapted to or configured to receive a spinal cord stimulator electrode (3) with an electrode lead (25) extending from the electrode sheath outlet end (11) for placement of the electrode lead (25) in the epidural space (6) of the subject (7) (as shown in the example of FIG. 4). In particular embodiments, an annular elastomeric member (38) can be integral to the electrode sheath internal surface (36) or disposed within the sheath interior space (37). The spinal cord stimulator electrode (3) can pass through the annular elastomeric member (38). The annular elastomeric member (38) can resiliently slidable engage the spinal cord stimulator electrode (3). The annular elastomeric member (38) can apply sufficient frictional force to allow movement of the spinal cord stimulator electrode (3) upon forceful urging by a practitioner while resisting movement of the spinal cord stimulator electrode (3) after placement of the spinal cord stimulator electrode (3) in the subject (7).

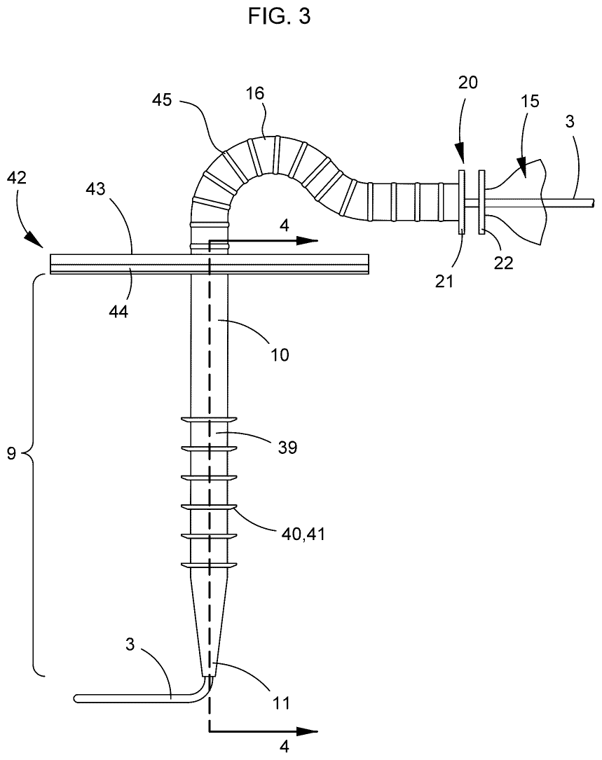

Now, with primary reference to FIGS. 3 and 4, the electrode sheath (9) can have an electrode sheath external surface (39) adapted to or configured to implant in the tissue disposed between the vertebrae (5) of the subject (7). While the illustrative examples depict a generally conical electrode sheath (9), this is not intended to preclude embodiments of the electrode sheath (9) that can be generally cylindrical, or another configuration that provides an appropriate purchase with the tissue upon implantation. In particular instances, the sheath external surface (39) can include implant stability elements (40) to resist movement of the electrode sheath (9) within the tissue of the subject (7) subsequent to implantation.

In the illustrative example of FIG. 3, the implant stability elements (40) comprise a plurality of annular projections (41) disposed in fixed spaced apart relation on the sheath external surface (39); however, this is not intended to preclude embodiments in which the implant stability element (40) comprises other configurations of raised or recessed features of the sheath external surface (39), including as illustrative examples: projections, protuberances, bumps, barbs, hooks, grooves, dimples, and indentations and combinations thereof.

The electrode sheath (9) can be produced from a biocompatible implantable grade material including as illustrative examples: a polystyrene, polypropylene, polyvinyl chloride, polyethylene, polyurethane, polycarbonate, polyethylene terephthalate, polyetheretherketone, polytetrafluoroethylene, ethylene tetrafluoroethylene, fluorinated ethylene propylene, perfluoroalkoxy, polyvinylidene difluoride, stainless steel, titanium, and nitinol, and combinations thereof.

Again, with primary reference to FIGS. 3 and 4, in particular embodiments, a sheath stabilizer (42) can be coupled to the sheath external surface (39). The sheath stabilizer (42) can attach to the subject (7) to resist egress of the electrode sheath (9) from the body of the subject (7). The sheath stabilizer (42) can comprise a rigid or flexible member extending from the sheath external surface (39) proximate the point of egress of the electrode sheath (9) from the body of the subject (7). In the illustrative example of FIG. 3, the sheath stabilizer (42) comprises a flexible annular member (43) disposed around the sheath external surface (39) having on the subject facing side of the flexible annular member (43) an sheath stabilizer adhesive layer (44) which can be adhered to the body of the subject (7) to resist egress of the implanted electrode sheath (9) from the body of the subject (7); however, this is not intended to preclude other methods of sheath stabilization including sutures, staples, closure bandages, adhesive tapes, and combinations thereof.

Again, with primary reference to FIGS. 2 through 9, in particular embodiments, a flexible conduit (16) can be integral with or coupled to the sheath inlet end (10). The flexible conduit (16) can extend between the sheath inlet end (10) of the electrode sheath (9) and the electrode sheath connector (20). In particular embodiments, an anti-crimp element (45) can be integral with or coupled to the flexible conduit (16) to resist folding, kinking of the spinal cord stimulator electrode (3). In the illustrative example of FIG. 3, the flexible conduit (16) includes an anti-crimp element (45) in the form of annular ribs (46) integrally formed in the flexible conduit (16) which engage to resist over bending of the flexible conduit (16); however, this illustrative embodiment is not intended to preclude other embodiments of an anti-crimp element (45) such as helical ribs and spaced apart coils.

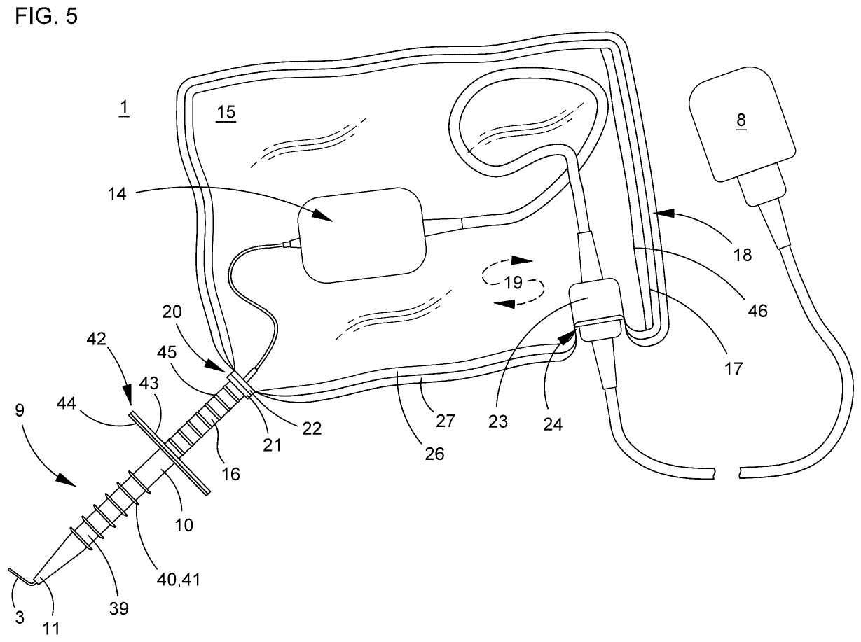

Now, with primary reference to FIGS. 5 through 7, particular embodiments of the apparatus (1) can further include, a pouch (15) coupled directly to the electrode sheath inlet end (10), or indirectly to sheath inlet end (10) by the flexible conduit (16), and extending to a pouch open end (18). The pouch (15) can be configured to enclose within a portion of the spinal cord stimulator electrode (3) extending from the electrode sheath inlet end (10) or the flexible conduit (16) and coupled to the electrically conductive interface (14). In particular embodiments, the pouch (15) can be affixed in immovable relation to the sheath inlet end (10) or to the flexible conduit (16). As examples, the affixation can be in the form of an adhesive bond, laser weld, friction fit and combinations thereof. In other embodiments, the pouch (15) can be removably affixed to the electrode sheath inlet (10) or the flexible conduit (16) by mateable connector parts (21, 22) with a first connector part (21) coupled to the electrode sheath inlet end (10) or the flexible conduit (16) and a second connector part (22) coupled to the pouch (15). The first connector part (21) removably mates to the second connector part (22) to couple the electrode sheath (9) to the pouch (15). Illustrative examples of mateable connector parts (21, 22) can take the form of a friction fit, a snap fit, a pair of mateable spiral threads, a luer lock, a male fitting and a female fitting, and compression fit. In particular embodiments, the pouch (15) can further include a pouch open end closure (46). The pouch open end closure (46) can irreversibly close the pouch open end (18) in the form of an applied adhesive, mateable adhesive layers, double sided adhesive strip, heat seal, laser weld, or the like. In other embodiments, the pouch open end closure (46) can releasably close the pouch open end (18) in the form of a fold over closure, zip fastener, binder clip, twist tie, hook and loop, as examples. In particular embodiments, the pouch (15) can further include a connector access port (24) adapted or configured to seal about a conductive interface connector (23). The pouch (15) can provide a sterile pouch internal surface (17) and upon closure of the pouch open end (18) can enclose a sterile interior space (19). As illustrative examples, the pouch (15) can be produced from polystyrene, polypropylene, polyvinyl chloride. polyethylene. polyurethane, polycarbonate, polyethylene terephthalate, polyetheretherketone, polytetrafluoroethylene, ethylene tetrafluoroethylene, fluorinated ethylene propylene, perfluoroalkoxy, polyvinylidene difluoride, and combinations thereof.

In particular embodiments, the pouch (15) can further include a pouch biocompatible adhesive layer (26). The pouch biocompatible adhesive layer (26) can be adapted or configured to adhere the pouch (15) to the subject (7) to fix the pouch (15) in fixed spatial relation to the electrode sheath (9) implanted in the subject (7). The pouch biocompatible adhesive layer (26) can be a biocompatible pressure sensitive adhesive layer with examples including: polyacrylics, polyurethanes, and silicones, and combinations thereof. The pouch biocompatible adhesive layer (26) can be covered with pouch biocompatible adhesive peelable layer (27) which can be peeled away from the pouch biocompatible adhesive layer (26).

Now, with primary reference to FIGS. 6 through 9, embodiments can further include a receptacle (29) having receptacle mateable portions (47, 48) engageable to define a receptacle external surface (49) which can be adapted to affix the receptacle (29) to a subject (7) proximate the implant location of the electrode sheath (9) and a receptacle internal surface (50) defining a receptacle interior space (51) extending between a receptacle inlet port (32) and a receptacle outlet port (30) each open between the receptacle internal surface (50) and the receptable external surface (49) (as shown in the example of FIG. 6). The receptacle interior space (50) can be adapted to or configured to receive the pouch (15) enclosing the spinal cord stimulator electrode (3) extending from the electrode sheath inlet end (10) and the electrically conductive interface (14). In particular embodiments, the pulse generator (8) can be disposed outside of the receptacle (29) and can be removably coupled to the electrically conductive interface (14) by a pulse generator cord (31) extending through the receptacle inlet port (32) (as shown in the example of FIG. 6). In particular embodiments, the pulse generator (8) can be disposed in the receptacle (29) (as shown in the example of FIG. 7). The pouch (15) coupled to the sheath inlet end (10) of the electrode sheath (9) or to the flexible conduit (16) can in part extend through the receptacle outlet port (30) to allow implantation of said electrode sheath (9) in the subject (7).

Again, with primary reference to FIGS. 6 through 9, the receptacle (29) can comprise mateable portions (47, 48) including a receptacle body (52) having a receptable bottom (53) joined to an upwardly extending receptacle sidewall (54) terminating in a receptacle rim (55) and a receptacle cover (56) configured to mateably affix to the receptacle rim (55). The receptacle cover (56) can include a hinge (57) integral with the receptacle body (52). The receptacle bottom (53) can include a resiliently flexible layer (58) compressible to contour to the anatomy of the subject (7) (as shown in the example of FIG. 9). The receptacle (29) can be produced from a wide variety of materials including ridged, flexible or foam materials, and combinations thereof, with illustrative examples including: polystyrene, polypropylene, polyvinyl chloride, polyethylene, polyurethane, polycarbonate, polyethylene terephthalate, polyetheretherketone, polytetrafluoroethylene, ethylene tetrafluoroethylene, fluorinated ethylene propylene, perfluoroalkoxy, polyvinylidene difluoride, latex foam, polyethylene foam, polyurethane foam, and combinations thereof.

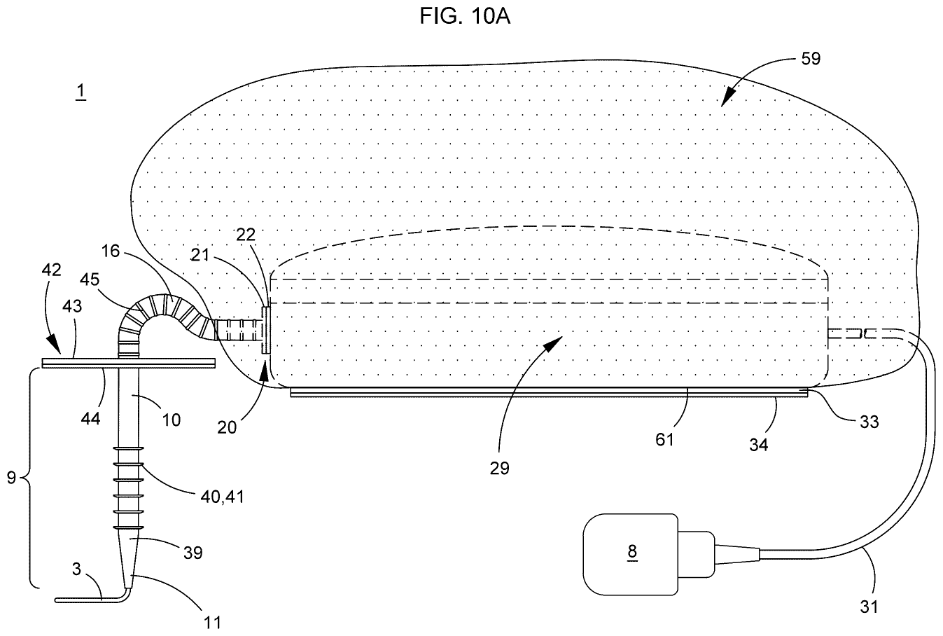

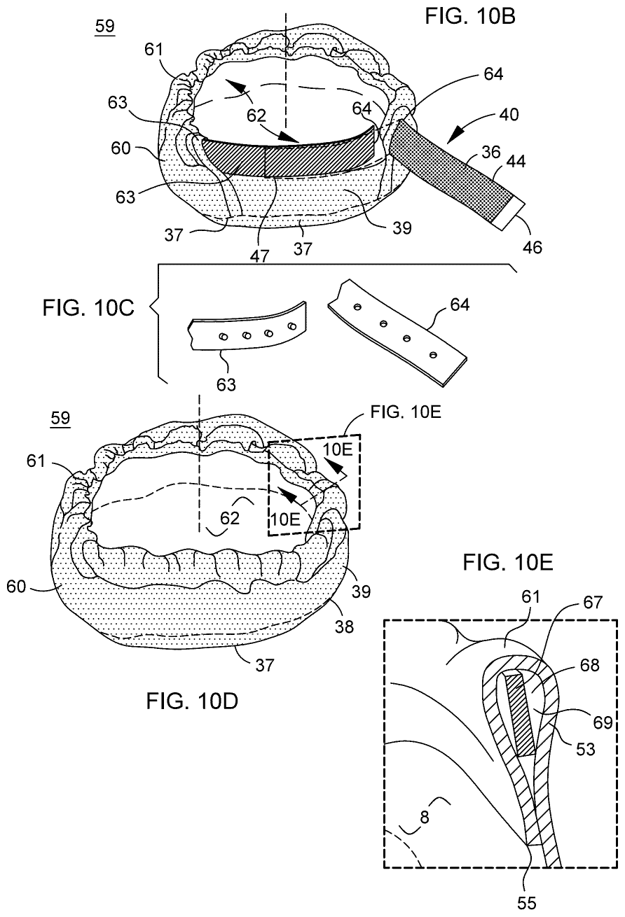

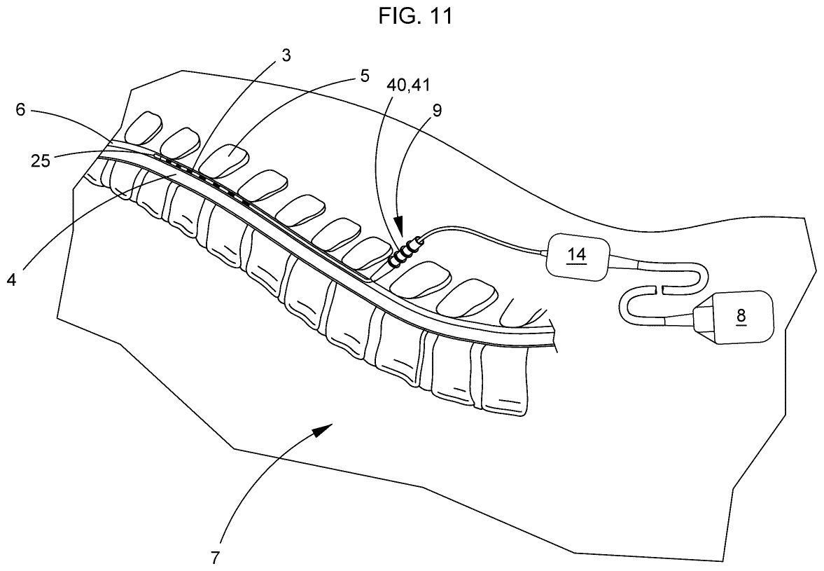

Now, with primary reference to FIG. 10A through 10E, embodiments can further include a cushion wrap (59) removably disposed about the receptacle (29) to absorb forces received by the receptacle (29). The cushion wrap (59), whether separate from, or as part of, the receptacle (29) can include a flexible cushion wrap body (60) which extends to a cushion wrap peripheral margin (61). The flexible cushion wrap body (60) can define a flexible body interior space (62) having a volume sufficient to surround the receptacle (29). The cushion wrap (59) can be secured or releasably secured to the receptacle (29). By way of illustrative example, the cushion wrap (59) can be secured or releasably affixed to the receptacle by: friction fit of the cushion wrap peripheral margin (61) to the receptacle external surface (50), mechanical fasteners, releasably mateable hook material and loop material, such as VELCRO®, elastic bands, or the combination thereof. The cushion wrap peripheral margin (61) of the cushion wrap (59) can, but need not necessarily, be substantially continuous or continuous and correspondingly continuously engage the receptacle (29). In particular embodiments, the cushion wrap peripheral margin (61) of the cushion wrap (59) can be interrupted or discontinuous and the cushion wrap perimeter margin (61) can engage the receptacle (29) at one or a plurality of attachment points. In the illustrative example of FIGS. 10B and 10C, the cushion wrap peripheral margin (61) can be discontinuous defining a distance between a pair of peripheral margin ends (63)(64). The peripheral margin ends (63)(64) can correspondingly define a first portion (65) releasably mateable to a second portion (66). The first portion (65) and the second portion (66) can be releasably mated by knottingly engagement, a first portion hook and second portion loop (as shown in the example of FIG. 10B), a snap back catch (as shown in the example of FIG. 10C), and combinations thereof.

In the illustrative examples of FIG. 10D and FIG. 10E, the cushion wrap (59) can, but need not necessarily include, an elastic element (67) coupled to or integral to the cushion wrap peripheral margin (61). The elastic element (67) can be elastically stretchable to allow the cushion wrap peripheral margin (61) of the cushion wrap (59) to be secured about the receptacle (29). In particular embodiments, the elastic element (67) can, but need not necessarily, be integral in the material used in the cushion wrap peripheral margin (61) of the cushion wrap (59), such as neoprene or materials such as SPANDEX® or LYCRA®. In particular embodiments, the elastic element (67) can, but need not necessarily, be a discrete element coupled to the surface of the cushion wrap peripheral margin (61) or disposed within a peripheral margin pocket (68). The peripheral margin pocket (68) can define a pocket interior space (69) sufficient to receive the elastic element (67). In particular embodiments, the material of the cushion wrap peripheral margin (61) can be folded and joined proximate a cushion wrap peripheral edge (70) to create the peripheral margin pocket (68) and the elastic element (67) can be drawn through the pocket interior space (69); however this illustrative example is not intended to preclude embodiments of the peripheral margin pocket (68) constructed by joining discrete pieces of material. The peripheral margin pocket (68) can extend the length of the cushion wrap peripheral margin (61).

Now, with primary reference to FIG. 11, which illustrates an embodiment of the apparatus (1) in which the electrode sheath (9) remains implanted in the subject (7) after completion of the spinal cord stimulation trial. In these instances, the portions of the electrode sheath (9) that are not implanted in the body of the subject (7) can be removed and the portions of the spinal cord stimulator (3) can be removed from the pouch (15) in a sterile condition ready to be implanted in the subject (7).

As can be easily understood from the foregoing, the basic concepts of the present invention may be embodied in a variety of ways. The invention involves numerous and varied embodiments of a spinal cord stimulator lead migration arrest and correction apparatus and methods for making and using such spinal cord stimulator lead migration arrest and correction apparatus including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “connector” should be understood to encompass disclosure of the act of “connecting”—whether explicitly discussed or not—and, conversely, were there is a disclosure of the act of “connecting”, such a disclosure should be understood to encompass disclosure of a “connector” and even a “means for connecting”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,”it will be understood that the particular element forms another embodiment.

Moreover, for the purposes of the present invention, the term “a” or “an” entity refers to one or more of that entity unless otherwise limited. As such, the terms “a” or “an”, “one or more” and “at least one”can be used interchangeably herein.

Further, for the purposes of the present invention, the term “coupled” or derivatives thereof can mean indirectly coupled, coupled, directly coupled, connected, directly connected, or integrated with, depending upon the embodiment.

Additionally, for the purposes of the present invention, the term “integrated” when referring to two or more components means that the components (i) can be united to provide a one-piece construct, a monolithic construct, or a unified whole, or (ii) can be formed as a one-piece construct, a monolithic construct, or a unified whole. Said another way, the components can be integrally formed, meaning connected together so as to make up a single complete piece or unit, or so as to work together as a single complete piece or unit, and so as to be incapable of being easily dismantled without destroying the integrity of the piece or unit.

Thus, the applicant(s) should be understood to claim at least: i) the spinal cord stimulator lead migration arrest and correction apparatus herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The elements following an open transitional phrase such as “comprising” may in the alternative be claimed with a closed transitional phrase such as “consisting essentially of” or “consisting of” whether or not explicitly indicated the description portion of the specification.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A spinal cord stimulator lead migration arrest device, comprising:
an electrode sheath implantable in an interspinal tissue of a subject, said electrode sheath having a sheath interior surface defining an interior space between a sheath inlet end and a sheath outlet end configured to receive a spinal cord stimulator electrode with one or more electrode leads extending from said sheath outlet end with placement of said one or more electrode leads in an epidural space of said subject; and
a pouch coupled to said sheath inlet end and extending to a pouch open end, said pouch configured to enclose within an electrically conductive interface coupled to said one or more spinal cord stimulator electrodes.

2. The device of claim 1, further comprising a receptacle having mateable portions engageable to define a receptacle exterior surface adapted to affix said receptacle to said subject proximate an implant location of said electrode sheath and a receptacle interior space extending between a receptacle inlet port and a receptacle outlet port open to said receptable exterior surface, said pouch enclosing said electrically conductive interface disposed within said receptacle, said pouch coupled to said sheath inlet end of said electrode sheath extending through said receptacle outlet port to allow implantation of said electrode sheath.

3. The device of claim 2, further comprising a cushion wrap removably disposed about said receptacle to absorb forces received by said receptacle.

4. The device of claim 1, further comprising a pulse generator disposed outside of said pouch, said pulse generator removably electrically coupled to said electrically conductive interface.

5. The device of claim 2, further comprising a pulse generator disposed outside of said receptacle, said pulse generator removably electrically coupled to said electrically conductive interface by a pulse generator cord extending through said receptacle inlet port.

6. The device of claim 1, further comprising an adhesive layer coupled to said pouch, said adhesive layer adapted to adhere said pouch to said subject to fix said pouch in fixed spatial relation to said electrode sheath implanted in said subject.

7. The device of claim 2, further comprising an adhesive layer coupled to said receptacle, said adhesive layer adapted to adhere said receptacle to said subject to fix said receptacle in fixed spatial relation to said electrode sheath implanted in said subject.

8. The device of claim 1, further comprising a sheath connector having mateable connector parts a first connector part coupled to said sheath outlet end and a second connector part coupled to said pouch, said first connector part removably mates to said second connector part to couple said electrode sheath to said pouch.

9. A method of making a spinal cord stimulator lead migration arrest device, comprising:
configuring an electrode sheath to implant in an interspinal tissue of a subject, said electrode sheath having a sheath interior surface defining an interior space between a sheath inlet end and a sheath outlet end configured to receive a spinal cord stimulator electrode with one or more electrode leads extending from said sheath outlet end for placement of said one or more electrode leads in an epidural space of said subject; and
coupling a pouch to said sheath inlet end and extending to a pouch open end, said pouch configured to enclose within an electrically conductive interface coupled to said one or more spinal cord stimulator electrodes.

10. The method of claim 9, further comprising configuring mateable portions of a receptacle which engage to define a receptacle exterior surface adapted to affix said receptacle to said subject proximate an implant location of said electrode sheath and a receptacle interior space extending between a receptacle inlet port and a receptacle outlet port open to said receptable exterior surface, said pouch enclosing said electrically conductive interface disposed within said receptacle, said pouch coupled to said sheath inlet end of said electrode sheath extending through said receptacle outlet port to allow implantation of said electrode sheath.

11. The method of claim 10, further comprising removably disposing a cushion wrap about said receptacle to absorb forces received by said receptacle.

12. The method of claim 9, further comprising disposing a pulse generator outside of said pouch, said pulse generator removably electrically coupled to said electrically conductive interface.

13. The method of claim 10, further comprising disposing a pulse generator disposed outside of said receptacle, said pulse generator removably electrically coupled to said electrically conductive interface by a pulse generator cord extending through said receptacle inlet port.

14. The method of claim 9, further comprising coupling an adhesive layer to said pouch, said adhesive layer adapted to adhere said pouch to said subject to fix said pouch in fixed spatial relation to said electrode sheath implanted in said subject.

15. The method of claim 10, further comprising coupling an adhesive layer to said receptacle, said adhesive layer adapted to adhere said receptacle to said subject to fix said receptacle in fixed spatial relation to said electrode sheath implanted in said subject.

16. The method of claim 9, further comprising coupling a first connector part to said sheath outlet end and coupling a second connector part to said pouch, said first connector part removably mates to said second connector part to couple said electrode sheath to said pouch.

17-27. (canceled)
